(19) United States
(12) Patent Application Publication
(10) Pub. No.: US 2026/0124252 A1
(43) Pub. Date: May 7, 2026

(54) Dietary Pattern Directed Nutritional Supplementation Composition and Methods for Attenuating Age-Related Cognitive Decline or Enhancing Cognitive Function

(71) Applicant: NeuroReserve Inc., Las Vegas, NV (US)

(72) Inventors: Edward S. Park, Las Vegas, NV (US); John F. O'Connell, Collegeville, PA (US); John C. Wallingford, Spokane, WA (US)

(21) Appl. No.: 19/238,574

(22) Filed: Jun. 16, 2025

Related U.S. Application Data
(60) Provisional application No. 63/660,752, filed on Jun. 17, 2024.

(51) Int. Cl. A61K 35/60; A61K 9/48; A61K 31/05; A61K 31/09; A61K 31/202; A61K 31/355; A61K 31/455; A61K 31/593; A61K 31/714; A61K 33/06; A61K 36/28; A61K 36/45; A61K 36/48; A61K 36/82

(52) U.S. Cl. CPC A61K 35/60; A61K 9/4808; A61K 31/05; A61K 31/09; A61K 31/202; A61K 31/355; A61K 31/455; A61K 31/593; A61K 31/714; A61K 33/06; A61K 36/28; A61K 36/45; A61K 36/48; A61K 36/82

(57) ABSTRACT

A functional food composition for attenuating age-related cognitive decline comprises multiple synergistic and additive nutrient components formulated to mimic Mediterranean and MIND dietary patterns. The composition includes omega-3 fatty acids in phospholipid form from herring roe oil, fat-soluble vitamins including cholecalciferol and tocopherols, carotenoids from marigold extract, B-vitamins including nicotinamide and hydroxocobalamin, magnesium bisglycinate chelate, polyphenolic compounds including catechins, flavonols, anthocyanins, and pterostilbene from various plant sources, and I-theanine. The composition achieves greater than 80% nutrient bioavailability through specialized delivery systems, including immediate-release capsules, delayed-release enteric capsules, and lipophilic softgels. Administration for 4-52 weeks to adults aged 50-90 years with suboptimal dietary patterns produces measurable improvements in biomarkers of cognitive health. A systematic method for developing such compositions based on dietary pattern analysis and multi-criteria nutrient scoring. Additionally, a method of inducing changes in important markers for brain aging and disease using a dietary pattern directed nutritional supplementation regimen.

### BACKGROUND OF THE INVENTION

### Field of Invention

The various aspects discussed herein relate to functional food and dietary supplement compositions for attenuating age-related cognitive decline and risk of age-related brain diseases.

### Description of Related Art

Age-related cognitive decline represents a significant health concern for adults, particularly those with suboptimal dietary patterns that lack adequate neuroprotective nutrients. The Mediterranean (MeDi) and Mediterranean-DASH Intervention for Neurodegenerative Delay (MIND) are often described as neuroprotective and containing neuroprotective nutrients. Observational studies have indicated that the MeDi and MIND diets are linked to reduced rate of age-related cognitive decline and reduced incidence of Alzheimer's disease (AD). Additionally, clinical trials investigating MeDi and MIND diets indicate benefits in cognitive function. The combination of nutrients delivered by MeDi and MIND diets is implicated with antioxidative, anti-inflammatory, lipid-lowering, and gut microbiota effects, and these in-turn may influence the pathophysiology of AD and cognitive decline.

However, a major problem is that adoption of MeDi or MIND diets and long-term adherence to such diets is difficult, particularly in the US and other countries where the “westernized” diet and lifestyle are prevalent. Barriers to these diets have included one or more of: cost, accessibility, personal habits and behavior, denial of disease risk, food preparation and cooking burden, family or household preferences, occupation and workplace, cultural norms, and other factors. Studies of US adults suggest significant intake gaps of foods and corresponding nutrients from MeDi and MIND diets that could support long-term brain health. For example, when comparing US dietary guidelines to average US adult intakes, there have been profound intake gaps for food groups associated with slower cognitive decline, such as, for example: berries (50% gap), leafy green vegetables (65% gap), fish (66% gap), and nuts and seeds (73%).

Additionally, existing approaches and scientific research in the nutritional products field often focus on individual nutrient supplementation or single botanical extracts, failing to capture the complex synergistic benefits observed with evidence-based dietary patterns, such as the MeDi and MIND diets. Conventional dietary supplement formulations typically do not replicate the proportional nutrient profiles found in these neuroprotective dietary patterns, nor do they address common intake gaps between high adherers of MeDi or MIND diets and average adult intakes, nor do they address the bioavailability challenges associated with delivering multiple nutrient classes in optimal ratios. Additionally, conventional supplement formulations typically are not designed as methods to affect concentrations (or levels) of certain nutrients, biomarkers, and metabolomic profiles that are indicators of brain aging, neurodegenerative diseases (e.g. Alzheimer's disease), or neuroprotective diets. Additionally, current methods for developing cognitive health supplements lack systematic approaches for translating epidemiological and clinical evidence from dietary pattern research into practical nutritional interventions.

Accordingly, there is a need in the art for functional food compositions that systematically mimic the nutrient profiles of evidence-based neuroprotective dietary patterns, such as MeDi and MIND, methods for developing such compositions, and methods applying such compositions to address age-related cognitive decline and risk of age-related brain diseases in adults with suboptimal diets.

### BRIEF SUMMARY OF THE INVENTION

This summary is provided to introduce a selection of concepts, in a simplified format, that are further described in the detailed description of the invention. This summary is neither intended to identify key or essential inventive concepts of the invention nor is it intended for determining the scope of the invention.

In one aspect, the present invention provides a functional food composition that solves the problems associated with conventional nutritional approaches to cognitive health by systematically delivering multiple nutrient classes in proportions that mimic evidence-based neuroprotective dietary patterns. The composition comprises an omega-3 fatty acid component consisting of docosahexaenoic acid and eicosapentaenoic acid in phospholipid form from herring roe oil, a fat-soluble vitamin component including cholecalciferol and tocopherols, a carotenoid component consisting of lutein and zeaxanthin from marigold flower extract, a B-vitamin component including nicotinamide and hydroxocobalamin, a mineral component consisting of magnesium as bisglycinate chelate, a polyphenolic component including catechins, quercetin, myricetin, kaempferol, anthocyanins, and pterostilbene from various botanical sources, and an amino acid component consisting of L-theanine, all formulated in pharmaceutically acceptable carriers as capsules and softgels.

Advantageously, the composition provides nutrients in proportions that mimic the nutritional profile of Mediterranean and MIND dietary patterns with at least 80% nutrient bioavailability, representing a significant improvement over existing formulations that fail to achieve such comprehensive nutrient delivery and absorption. The present invention improves upon the prior art by utilizing specific nutrient forms and delivery systems that enhance bioavailability, including phospholipid-bound omega-3 fatty acids that provide superior plasma omega-3 index or plasma omega-3 (DHA+EPA) increases compared to baseline, and specialized encapsulation technologies that preserve nutrient stability and optimize absorption.

In operation, daily consumption for 4-12 weeks by adults aged 50-90 years with suboptimal dietary patterns results in measurable improvements in plasma biomarkers of cognitive health, including increased 25-hydroxyvitamin D, increased omega-3 index, increased plasma omega-3 (DHA+EPA), increased lutein and zeaxanthin levels, and decreased neuroinflammatory markers. The composition may further comprise a multi-component delivery system with immediate-release capsules, delayed-release enteric capsules, or lipophilic softgels to optimize nutrient stability and absorption timing.

In another aspect, the present invention provides a systematic method for developing dietary pattern directed nutritional supplements for cognitive health that addresses the current lack of evidence-based approaches in supplement development. The method comprises identifying neuroprotective dietary patterns through systematic literature analysis, selecting target food groups, generating candidate nutrient lists, applying multi-criteria scoring systems, and determining intake levels based on diet-achievable concentrations that correspond to high-adherence Mediterranean and MIND dietary patterns.

In another aspect, the present invention provides a dietary pattern directed nutritional supplementation method to induce changes or differences in: (A) the concentrations of nutrients and metabolites (in blood, urine, tissue, or otherwise derived from human samples) indicative of nutritional components abundant in neuroprotective diets, such as the MeDi or MIND diets; (B) the concentrations of biomarkers of brain aging, Alzheimer's disease, or neurodegeneration or ratios of such biomarkers; (C) metabolomic profiles in a manner that resembles metabolomic profiles of high adherers of neuroprotective diets; (D) certain standard clinical laboratory blood measurements associated with cardiovascular, metabolic, or other aspects of health; (E) scores of standardized cognitive tests; (F) brain structure or imaging results; or (G) combinations of the above (A)-(F) in a manner indicating correlations between or among them. Such dietary pattern directed nutritional supplementation method may include a dietary supplement that is specifically designed to close nutrient gaps in the diet so that it may better resemble the nutritional intake profile of a neuroprotective diet, such as the MeDi or MIND diets. The supplement, for example, can consist of nutrients, such as 17 nutrients, abundant in MeDi or MIND diets, including one or more of certain vitamins, bioactive compounds, fatty acids, amino acids, and minerals.

The present invention has the advantage of providing a scientifically rigorous framework for translating epidemiological and clinical evidence from dietary pattern research into practical nutritional interventions, enabling the development of supplements that provide 12-25 distinct nutrients at nutritive intake levels equivalent to consuming evidence-based neuroprotective dietary patterns. This systematic approach represents a pioneering advancement in the field of cognitive health supplementation, offering the potential to improve quality of life for aging adults while providing a scalable methodology for developing evidence-based nutritional interventions across various therapeutic areas.

Additional features and advantages of the invention will be set forth in the description which follows. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 is a process-flow diagram illustrating a method 100 for developing a dietary pattern directed nutritional supplement for cognitive health through systematic nutrient selection and formulation optimization, in accordance with one embodiment.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word “or” as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

As used herein, the terms “functional food” or “functional food composition” are intended to include food, dietary supplement, or nutraceutical compositions.

As used herein, the term “attenuating age-related cognitive decline” includes outcomes that maintain, reduce or slow, or reverse: the rate of age-related cognitive decline (assessed by patient-reported outcomes, subjective assessments, or objective assessments via cognitive tests), the risk of age-related neurodegenerative diseases (e.g. Alzheimer's disease, Parkinson's disease, Lewy body dementia), the progression of brain aging processes or pathologies, the progression of neurodegenerative processes or pathologies, or the levels (or progression) of biomarkers of brain aging or neurodegeneration (where elevated levels indicate greater risk or progression toward cognitive decline or neurodegenerative disease). The term “attenuating age-related cognitive decline” also includes improvement in cognitive abilities or performance, as assessed by patient-reported outcomes, subjective assessments, or objective assessments via cognitive tests.

As used herein, the naming or listing of any polyphenolic substances, individually or as classes, such as “catechins,” “quercetin,” “kaempferol,” “myricetin,” or “anthocyanins,” includes the aglycone form of such polyphenolic substances and their derivatives (such as their glycoside forms).

The functional food composition comprises a strategically formulated multi-component nutraceutical system designed for attenuating age-related cognitive decline through synergistic or additive nutritional intervention. The composition is a daily dosing regimen that integrates seven distinct bioactive component categories arranged in a therapeutically effective matrix: an omega-3 fatty acid component, a fat-soluble vitamin component, a carotenoid component, a B-vitamin component, a mineral component, a polyphenolic component, and an amino acid component, all incorporated within a pharmaceutically acceptable carrier system.

The omega-3 fatty acid component consists of premium-grade herring roe oil (Clupea harengus), primarily sourced from North Atlantic fisheries, processed via supercritical CO₂ extraction at 35° C. and 300 bar pressure to preserve phospholipid integrity. The herring roe oil is standardized to contain ≥70 wt % phospholipid-bound omega-3 fatty acids with docosahexaenoic acid (DHA) content of 200-800 mg per dosage unit and eicosapentaenoic acid (EPA) content of 58-232 mg per dosage unit, maintaining a DHA:EPA weight ratio of 2.75:1 to 3.25:1, as determined by gas chromatography-mass spectrometry (GC-MS) analysis.

The fat-soluble vitamin component incorporates cholecalciferol (vitamin D3) at concentrations of 1200-4800 IU, sourced from lanolin-derived precursors and purified to ≥98% purity by HPLC analysis or sourced from concentrated cod liver oil. The tocopherol fraction comprises RRR-alpha-tocopherol at 20-80 mg and gamma-tocopherol at 40-160 mg, derived from non-GMO soybean or sunflower oil through molecular distillation, maintaining tocopherol acetate ester stability with ≤0.5% oxidative degradation products.

The carotenoid component utilizes marigold flower extract (Tagetes erecta) standardized through hexane extraction and saponification, providing lutein at 5-20 mg and zeaxanthin at 1-4 mg in their naturally occurring 5:1 weight ratio. The extract contains ≥20% meso-zeaxanthin and maintains total carotenoid content at ≥90% by UV-Vis spectrophotometry at 445 nm.

The B-vitamin component incorporates nicotinamide (vitamin B3) at broad ranges of 25-400 mg, intermediate ranges of 40-300 mg, and preferred ranges of 50-200 mg per dosage unit. Hydroxocobalamin (vitamin B12) is included at broad ranges of 10-1000 mcg, intermediate ranges of 15-200 mcg, and preferred ranges of 20-100 mcg, including sourcing from bacterial fermentation with ≥98% purity by microbiological assay.

The mineral component comprises magnesium bisglycinate chelate providing elemental magnesium at broad ranges of 25-600 mg, intermediate ranges of 40-400 mg, and preferred ranges of 50-200 mg. The bisglycinate chelation maintains ≥90% solubility at physiological pH 7.4 and exhibits stability constants (log K) of 7.2-7.8 for optimal bioavailability enhancement.

The polyphenolic component represents a standardized phytochemical concentrate comprising catechins from Camellia sinensis at 160-800 mg with ≥40 wt % epigallocatechin gallate (EGCG) content, quercetin (a flavonol) at 50-200 mg, myricetin (a flavonol) at 10-40 mg, kaempferol (a flavonol) at 20-80 mg, anthocyanins from European blueberry Vaccinium myrtillus at 50-200 mg standardized to >25% anthocyanin content within extract, and pterostilbene from Pterocarpus marsupium at 25-100 mcg with ≥90% trans-isomer purity.

The composition is manufactured through a controlled multi-stage process involving component-specific preparation protocols. The polyphenolic components undergo convection-drying, freeze-drying, or spray-drying at inlet temperatures of 35° C. ±2° C. and outlet temperatures of 25° C.±1° C. under nitrogen atmosphere to preserve aglycone forms and maintain polyphenol oxidase inhibition values ≤5%. The carotenoids are in concentrated oil form or microencapsulated using a protein-polysaccharide matrix system through fluid-bed coating at 45° C. with 15% w/w coating solution application rate, achieving particle size distribution of 150-250 μm median diameter.

The lipophilic components including omega-3 fatty acids, fat-soluble vitamins, and carotenoids are formulated into softgel matrices using gelatin capsules shells. The remaining components, including B-vitamins and minerals are granulated using chilsonation and combined with l-leucine in hypromellose two-piece capsule shells.

The final composition exhibits specific physical-chemical properties optimized for bioavailability and stability. The softgel components demonstrate viscosity of 800-1200 cP at 25° C., with dissolution profiles showing ≥85% release within 30 minutes in simulated gastric fluid (SGF) at pH 1.2. The enteric-coated capsules containing polyphenolic components maintain gastric resistance for >2 hours at pH 1.2 and exhibit rapid disintegration within 15 minutes upon exposure to simulated intestinal fluid (SIF) at pH 6.8.

Particle size analysis reveals median diameters (D₅₀) of 10-180 μm for powder components with polydispersity indices ≤0.3. Moisture content is maintained at ≤3% w/w for powder components and ≤0.5% for lipophilic matrices. Zeta potential measurements for encapsulated carotenoids range from −15 to −25 mV, indicating colloidal stability.

In vitro assays demonstrate the composition's neuroprotective efficacy through multiple validated endpoints. Amyloid-β₁₋₄₂ aggregation inhibition assays using thioflavin-T fluorescence indicate IC₅₀ values of 180-220 μM for the complete polyphenolic component, representing synergistic interaction indices of <0.90 by Chou-Talalay analysis compared to individual component effects.

Neuroinflammation modulation is evidenced through primary microglial cell culture assays, where the composition demonstrates IL-6 cytokine reduction of 25-65% and TNF-α suppression of 15-55% compared to lipopolysaccharide-stimulated controls. Synaptic plasticity enhancement is validated through hippocampal slice preparations showing long-term potentiation (LTP) amplitude restoration to 65-95% of baseline values in aged tissue models.

The phospholipid-bound omega-3 formulation provides plasma omega-3 index increases or plasma omega-3 (DHA+EPA) increases of 1.25-2.2-fold compared to baseline, with peak plasma concentrations (Cₘ_{^{ax}}) achieved at 4-6 hours post-administration. The lipophilic vitamin matrix demonstrates bioavailability enhancement of ≥40% for tocopherols compared to dry powder encapsulation, as measured by plasma alpha-tocopherol/cholesterol ratio improvements.

Carotenoid bioavailability from the microencapsulated matrix shows lutein plasma concentrations increasing 2.1-2.8-fold compared to baseline within 4-6 weeks of administration. The magnesium bisglycinate chelate exhibits elemental magnesium bioavailability ≥25% higher than conventional magnesium oxide, with plasma magnesium levels reaching steady-state within 2-3 weeks.

In controlled human studies involving adults aged 50-90 years with Mediterranean-MIND dietary screener scores ≤7.5 out of 14, daily administration of the composition for 4-12 weeks produces statistically significant improvements in multiple plasma biomarkers. Specifically, 25-hydroxyvitamin D levels increase by >20%, omega-3 index (or plasma omega-3 (DHA+EPA) improves by 25%, lutein+zeaxanthin concentrations rise by >25%, and neuroinflammatory marker IL-6 decreases by >20% compared to baseline measurements.

Advanced metabolomic analysis reveals upregulation of 29 metabolites in NAD⁺ biosynthesis pathways and neuroprotective (or brain-aging) biochemical cascades. Plasma p-tau217 levels demonstrate reductions of >5%.

The composition incorporates a precision dosage stratification protocol based on baseline biomarker status. Adults presenting with 25-hydroxyvitamin D levels <30 ng/ml receive upper dosage ranges across all components (20-80), while individuals with adequate baseline nutrient status receive intermediate dosage ranges to maintain optimal plasma concentrations without exceeding tolerable upper intake levels.

The multi-component delivery system comprises immediate-release or delayed-release capsules containing B-vitamin and mineral components, delayed-release enteric capsules housing polyphenolic components with pH-dependent release mechanisms, and lipophilic softgels incorporating omega-3, vitamin, and carotenoid components. This multi-modal approach ensures optimal absorption timing and minimizes potential component interactions.

Accelerated aging studies conducted at 40° C./75% relative humidity demonstrate ≥90% potency retention for all active components over 24 months storage at 25° C./60% relative humidity. The microencapsulated carotenoids maintain color stability and antioxidant activity with ≤5% degradation under fluorescent light exposure conditions. Softgel matrices exhibit ≤2% moisture uptake and maintain seal integrity under thermal cycling conditions.

Primary packaging utilizes aluminum-polyethylene laminate blister packs or polyethylene terephthalate (PET) containers with oxygen transmission rates ≤0.1 cc/m²/day and moisture vapor transmission rates ≤0.05 g/m²/day. Desiccant sachets containing molecular sieve beads maintain internal relative humidity ≤30% throughout shelf life.

The composition demonstrates full industrial applicability through validated scale-up protocols utilizing current Good Manufacturing Practice (cGMP) facilities.

The formulation strategy mimics Mediterranean dietary pattern adherence scores ≥8 out of 14 while providing ≥80% nutrient bioavailability through advanced delivery technologies, establishing utility as a therapeutically effective dietary intervention for cognitive health maintenance in aging populations with suboptimal nutritional intake patterns.

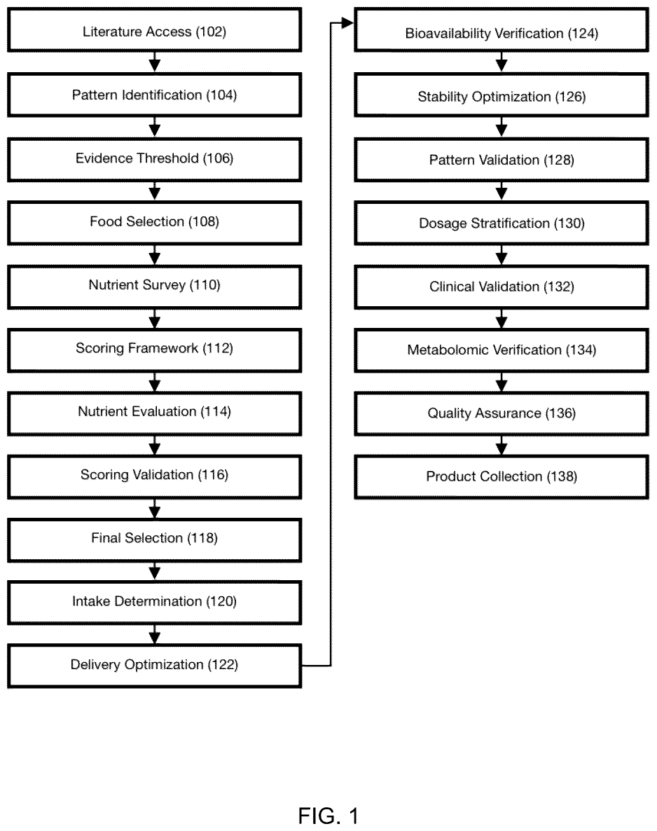

FIG. 1 is a process-flow diagram illustrating a method 100 for developing a dietary pattern directed nutritional supplementation regimen for cognitive health through systematic nutrient selection and formulation optimization, in accordance with one embodiment.

The method 100 initiates at step 102 with providing comprehensive database access to peer-reviewed human studies examining Mediterranean diet and MIND diet adherence protocols, wherein literature search parameters encompass cognitive outcomes, neuroprotective evidence, and dietary pattern scoring methodologies under HACCP-validated research conditions. Subsequently, step 104 involves analyzing systematic literature data to identify neuroprotective dietary patterns through quantitative meta-analysis techniques, scoring each dietary pattern based on strength of neuroprotective evidence derived from longitudinal cognitive assessment studies spanning ≥5 years follow-up periods.

The process flow proceeds to decision diamond 106, which determines whether dietary pattern neuroprotective evidence scores exceed a predetermined threshold of ≥7.5 on a standardized cognitive benefit scale. If this threshold is met, branch A proceeds to food group selection at step 108; otherwise, branch B returns to pattern re-evaluation via return line R1 to step 104. When proceeding along branch A, step 108 involves selecting target food groups abundant in highest-scoring neuroprotective dietary patterns, said food groups including fish providing phospholipid-bound omega-3 fatty acids, leafy green vegetables rich in folate and lutein, berries containing anthocyanins, nuts supplying alpha-tocopherol, tea providing catechins, and olive oil contributing monounsaturated fatty acids.

The methodology advances to step 110, which encompasses generating a candidate nutrient list through comprehensive literature survey encompassing nutrients abundant in selected food groups and studied in context of cognition, neuroprotection, or neurodegeneration, with particular emphasis on bioavailable forms and synergistic combinations. This nutrient identification process transitions into step 112, wherein multi-criteria nutrient scoring methodology is applied using five evaluation criteria consisting of observational/epidemiological evidence strength for cognitive benefit, clinical evidence quality and cognitive outcomes, putative biological mechanisms categorized by neuroprotective pathways, likelihood of intake inadequacy in target populations with suboptimal diets, and safety profile with established upper intake limits.

Step 114 involves evaluating each candidate nutrient through systematic scoring across all five criteria using weighted algorithms, wherein herring roe oil comprising ≥70 wt % phospholipid-bound omega-3 fatty acids with DHA:EPA weight ratio of 2.75:1 to 3.25:1 receives enhanced bioavailability scoring, and wherein RRR-alpha-tocopherol and gamma-tocopherol co-formulation demonstrates superior plasma elevation profiles. The process then reaches decision diamond 116, which determines whether total nutrient scores across all criteria meet minimum threshold requirements and represent diverse food groups with distinct neuroprotective mechanisms. Upon meeting these validation criteria, branch B advances to final selection at step 118; otherwise, branch A returns to scoring refinement via return line R2 to step 114.

Final nutrient selection occurs at step 118 by ranking candidates by total scores across all criteria and choosing top-scoring nutrients representing different food groups and diverse neuroprotective mechanisms, including marigold flower extract standardized to lutein:zeaxanthin 5:1 weight ratio with ≥20% meso-zeaxanthin and polyphenolic components. The process continues to step 120, which involves determining intake levels for selected nutrients based on diet-achievable concentrations below established upper limits corresponding to proportions found in high-adherence Mediterranean and MIND dietary patterns, wherein magnesium bisglycinate chelate exhibits ≥75% solubility at physiological pH.

Formulation optimization commences at step 122 with developing a multi-component delivery system comprising immediate-release capsules or delayed release containing B-vitamin and mineral components, delayed-release enteric capsules containing polyphenolic components with gastric resistance ≥2 hours at pH 1.2, and lipophilic softgels containing omega-3, vitamin, and carotenoid components. Subsequently, step 124 involves confirming that phospholipid omega-3 form provides plasma omega-3 index increase or plasma omega-3 (DHA+EPA) increase ≥1.25-fold compared to baseline, and that RRR-alpha-tocopherol/gamma-tocopherol lipophilic matrix provides ≥40% bioavailability enhancement compared to dry powder encapsulation as measured by plasma alpha-tocopherol/cholesterol ratio.

Stability optimization occurs at step 126 through microencapsulating carotenoids in protein-polysaccharide matrix exhibiting storage stability ≥90% potency retention at 25° C./60% RH for 24 months, and processing catechins to comprise ≥40 wt % epigallocatechin gallate (EGCG) with polyphenol oxidase inhibition value ≤5%. The formulation process then advances to decision diamond 128, which evaluates whether nutrient ratios mimic Mediterranean dietary pattern adherence scores ≥8 out of 14 and demonstrate synergistic neuroprotective activity with ≥5% greater reduction in plasma p-tau217 levels compared to baseline. Upon meeting these validation criteria, branch B proceeds to dosage stratification at step 130; otherwise, branch A returns to ratio adjustment via return line R3 to step 120.

Dosage stratification implementation occurs at step 130, wherein adults with baseline 25-hydroxyvitamin D levels <30 ng/ml receive upper dosage ranges of all components, ensuring systematic development of nutritional supplements providing 12-25 distinct nutrients at nutritive intake levels equivalent to consuming at least 2-3 weekly servings of fish, daily leafy greens, daily nuts, and daily tea consumption. The process advances to step 132, which establishes clinical validation protocol wherein daily administration for 4-12 weeks to adults aged 50-90 years with MIND dietary screener scores ≤7.5 produces statistically significant (p≤0.05) or near statistically significant (p≤0.10) improvements (by comparing measurements at baseline vs time points after daily administration is applied (or the reverse: comparing measurements while daily administration is applied vs the removal of daily administration) or by comparing measurements between daily administration and control groups) in at least three biomarkers (derived from blood or urine) selected from: omega-3 (docosahexaenoic acid (DHA), eicosapentaenoic acid (EPA), or DHA+EPA), omega-3 index, plasma omega-3 (DHA+EPA), total 25-hydroxyvitamin D (25 (OH) D), alpha-and gamma-tocopherol (vitamin E forms), lutein, zeaxanthin, nicotinamide (vitamin B3), cobalamin (vitamin B12 and other vitamin B forms), magnesium, catechins (including epigallocatechin gallate (EGCG), epigallocatechin (EGC), and epicatechin (EC)), quercetin, kaempferol, myricetin, anthocyanins (including parent anthocyanidins, which include cyanidin, delphinidin, malvidin, pelargonidin, peonidin, petunidin).

Metabolomic pathway verification proceeds at step 134 through conducting plasma metabolomic analysis after 12 weeks administration to confirm upregulation of ≥9 metabolites in NAD+ biosynthesis, metabolites upregulated in high adherers to the MIND or MeDi diets, or neuroplasticity pathways compared to control, demonstrating that supplements developed by this method systematically mimic complex nutrient profiles of evidence-based neuroprotective dietary patterns rather than targeting individual nutrient deficiencies or single botanical extracts. The method concludes with step 136, which involves verifying FDA 21 CFR Part 111 compliance for dietary supplement manufacturing under GMP conditions, followed by step 138, which collects the finalized dietary pattern directed nutritional supplementation regimen for commercial distribution to target populations with suboptimal dietary adherence patterns.

The functional food composition described in this detailed description also introduces a novel method, comprising use of a dietary pattern directed nutritional supplementation regimen, to affect the concentrations (or levels) of biological markers, alone and in combination, implicated in brain aging and brain age-related diseases. The effects induced by this method include changes or differences in:
(A) the concentrations of nutrients and metabolites (in blood, urine, tissue, or otherwise derived from human samples) indicative of nutritional components abundant in neuroprotective diets, such as the MeDi or MIND diets, including:
   Omega-3 (docosahexaenoic acid (DHA), eicosapentaenoic acid (EPA), DHA+EPA);
   Total 25-hydroxyvitamin D (25(OH)D);
   Alpha-and gamma-tocopherol (E);
   Lutein and zeaxanthin;
   Nicotinamide (B3);
   Cobalamin (B12);
   Magnesium;
   Catechins (including epigallocatechin gallate (EGCG), epigallocatechin (EGC), and epicatechin (EC));
   Quercetin;
   Kaempferol;
   Myricetin;
   Anthocyanins (across parent anthocyanidins including: cyanidin, delphinidin, malvidin, pelargonidin, peonidin, petunidin);
(B) the concentrations of biomarkers of brain aging, Alzheimer's disease, or neurodegeneration, including ptau-217, ptau-181, total tau, amyloid beta 42 (Aβ42), amyloid beta 40 (Aβ40), glial fibrillary acidic protein (GFAP), neurofilament light chain (NfL), pro-and anti-inflammatory cytokines implicated in brain aging and a number of neurodegenerative diseases as listed in Table 1, or ratios of such biomarkers (including ptau-271/Aβ42);

TABLE 1
Pro- and Anti-Inflammatory Cytokines

| | | | |
|---|---|---|---|
| Basic FGF | IL-2 | IL-13 | MIP-1alpha |
| CTACK | IL-2Ralpha | IL-15 | MIP-1beta |
| Eotaxin | IL-3 | IL-16 | beta-NGF |
| G-CSF | IL-4 | IL-17A | PDGF-BB |
| GM-CSF | IL-5 | IL-18 | RANTES |
| GRO-alpha | IL-6 | IP-10 | SCF |
| HGF | IL-7 | LIF | SCGF-beta |
| IFN-alpha2 | IL-8 | MCP-1(MCAF) | SDF-1alpha |
| IFN-gamma | IL-9 | MCP-3 | TNF-alpha |
| IL-1alpha | IL-10 | M-CSF | TNF-beta |
| IL-1beta | IL-12(p70) | MIF | TRAIL |
| IL-1Ra | IL-12(p40) | MIG | VEGF |

(C) metabolomic (metabolic), lipidomic, proteomic, exposomic, or ionomic profiles in a manner that more closely resembles the respective profiles of high adherers of neuroprotective diets (e.g. MeDi or MIND), measured through either targeted or unbiased analyses, where metabolomic analyses may cover amino acid, cofactor/vitamin, nucleotide, carbohydrate, energy, lipid, and other metabolic classes;
(D) certain standard clinical laboratory blood measurements associated with cardiovascular, metabolic, or other aspects of health associated with long-term brain health, including LDL, HDL, triglycerides, total cholesterol, fasting glucose, HbA1C, and CRP;
(E) scores of standardized cognitive tests, which may include rapid (3-15 min), self-administered digital/computerized cognitive screening tests, where cognitive domains tested may include memory, executive function, attention, visuospatial, and processing speed;
(F) brain structure or imaging results, such as brain size, volume, specific area density, phosphorylated tau levels (for example, through positron-emission tomography (PET), brain amyloid plaque levels, brain volume or connectivity in total or in specific regions, gray matter, or white matter; or
(G) combinations of the above (A)-(F) indicating correlations between or among them. Changes or differences in items (A)-(F) could be derived by comparing measurements at baseline vs time points after the method is applied (or the reverse: comparing measurements while method is applied vs the removal of method) or by comparing measurements between method and control groups.

The effects of the aforementioned dietary pattern directed nutritional supplementation method may be demonstrated through data derived from novel human studies or clinical trials, investigating such method alone or in combination with other lifestyle interventions. Such human studies or clinical trials may involve the measurement of (A) nutrients, (B) biomarkers of brain aging, Alzheimer's disease, or neurodegeneration, (C) multi-omics profiles, (D) certain standard laboratory measures, (E) standardized cognitive assessments, and (F) brain structure through imaging or other means, as described above, which could be derived from from blood, urine, cerebrospinal fluid (CSF), tissue specimens, human subject responses, and imaging data.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A functional food composition for attenuating age-related cognitive decline in adults with suboptimal dietary patterns, comprising a daily regimen of:
a) omega-3 fatty acid component consisting of 200-800 mg docosahexaenoic acid (DHA) and 58-232 mg eicosapentaenoic acid (EPA) in phospholipid form from herring roe oil;
b) fat-soluble vitamin component consisting of 1200-4800 IU cholecalciferol (vitamin D3), 20-80 mg RRR-alpha-tocopherol, and 40-160 mg gamma-tocopherol;
c) carotenoid component consisting of 5-20 mg lutein and 1-4 mg zeaxanthin from marigold flower extract;
d) B-vitamin component consisting of 50-200 mg nicotinamide (vitamin B3) and 20-100 mcg hydroxocobalamin (vitamin B12);
e) mineral component consisting of 50-200 mg elemental magnesium as bisglycinate chelate;
f) polyphenolic component consisting of 160-800 mg catechins from Camellia sinensis, 50-200 mg quercetin (a flavonol), 10-40 mg myricetin (a flavonol), 20-80 mg kaempferol (a flavonol), 50-200 mg anthocyanins from European blueberry (Vaccinium myrtillus), and 25-100 mcg pterostilbene from Pterocarpus marsupium;
g) amino acid component consisting of 50-200 mg L-theanine; and
h) a pharmaceutically acceptable carrier formulated as capsules and softgels for once or twice daily administration, wherein the composition provides nutrients in proportions that mimic the nutritional profile of nutrients characteristic of MeDi and MIND dietary patterns.

2. The composition of claim 1, wherein the herring roe oil comprises ≥70 wt % phospholipid-bound omega-3 fatty acids with a DHA:EPA weight ratio of 2.75:1 to 3.25:1, and wherein the phospholipid form provides a plasma omega-3 index increase or plasma omega-3 (DHA+EPA) increase of at least 1.25-fold compared to baseline.

3. The composition of claim 1, wherein the RRR-alpha-tocopherol and gamma-tocopherol are co-formulated in a lipophilic softgel, providing a bioavailability enhancement of ≥40% compared to dry powder encapsulation as measured by plasma alpha-tocopherol/cholesterol ratio.

4. The composition of claim 1, wherein the marigold flower extract is standardized to provide lutein and zeaxanthin in their naturally occurring 5:1 weight ratio with ≥20% as meso-zeaxanthin.

5. The composition of claim 1, wherein the polyphenolic components are processed via convection-drying, freeze-drying, or spray-drying at temperatures ≤40° C. to preserve bioactive aglycone forms, and wherein the catechins comprise ≥40 wt % epigallocatechin gallate (EGCG) with a polyphenol oxidase inhibition value ≤5%.

6. The composition of claim 1, formulated as a multi-component delivery system comprising:
a) immediate-release or delayed-release capsules containing the B-vitamin and mineral components,
b) delayed-release enteric capsules containing the polyphenolic components with gastric resistance for ≥2 hours at pH 1.2, and
c) lipophilic softgels containing the omega-3, vitamin, and carotenoid components.

7. The composition of claim 1, wherein daily administration for 4-12 weeks to adults aged 50-90 years with MIND dietary screener scores ≤7.5 produces statistically significant (p≤0.05) or near statistically significant (p≤0.10) improvements (by comparing measurements at baseline vs time points after daily administration is applied (or the reverse: comparing measurements while daily administration is applied vs the removal of daily administration) or by comparing measurements between daily administration and control groups) in at least three biomarkers (derived from blood or urine) selected from: omega-3 (docosahexaenoic acid (DHA), eicosapentaenoic acid (EPA), or DHA+EPA), omega-3 index, plasma omega-3 (DHA+EPA), total 25-hydroxyvitamin D (25 (OH) D), alpha-and gamma-tocopherol (vitamin E forms), lutein, zeaxanthin, nicotinamide (vitamin B3), cobalamin (vitamin B12 and other vitamin B forms), magnesium, catechins (including epigallocatechin gallate (EGCG), epigallocatechin (EGC), and epicatechin (EC)), quercetin, kaempferol, myricetin, anthocyanins (including parent anthocyanidins, which include cyanidin, delphinidin, malvidin, pelargonidin, peonidin, petunidin);

8. The composition of claim 1, wherein daily administration for 4-52 weeks to adults aged 50-90 years with MIND dietary screener scores ≤7.5 produces statistically significant (p≤0.05) or near statistically significant (p≤0.10) improvements (by comparing measurements at baseline vs time points after daily administration is applied (or the reverse: comparing measurements while daily administration is applied vs the removal of daily administration) or by comparing measurements between daily administration and control groups) in at least one biomarker (derived from blood or cerebrospinal fluid) selected from: ptau-217, ptau-181, total tau, amyloid beta 42 (Aβ42), amyloid beta 40 (Aβ40), glial fibrillary acidic protein (GFAP), neurofilament light chain (NfL), or pro-and anti-inflammatory cytokines implicated in brain aging and a number of neurodegenerative diseases (including (in their common scientific abbreviations) basic FGF, CTACK, eotaxin, G-CSF, GM-CSF, GRO-alpha, HGF, IFN-alpha2, IFN-gamma, IL-1alpha, IL-1beta, IL-1Ra, IL-2, IL-2Ralpha, IL-3, IL-4, IL-5, IL-6, IL-7, IL-8, IL-9, IL-10, IL-12 (p70), IL-12 (p40, IL-13, IL-15, IL-16, IL-17A, IL-18, IP-10, LIF, MCP-1 (MCAF), MCP-3, M-CSF, MIF, MIG, MIP-1alpha, MIP-1beta, beta-NGF, PDGF-BB, RANTES, SCF, SCGF-beta, SDF-1alpha, TNF-alpha, TNF-beta, TRAIL, and VEGF).

9. The composition of claim 1, wherein daily administration for 4-52 weeks to adults aged 50-90 years with MIND dietary screener scores ≤7.5 produces statistically significant (p≤0.05) or near statistically significant (p≤0.10) improvements (by comparing measurements sat baseline vs time points after daily administration is applied (or the reverse: comparing measurements while daily administration is applied vs the removal of daily administration) or by comparing measurements between daily administration and control groups) to the ratio of blood-derived biomarkers ptau-217 and Aβ42 (i.e. ratio of ptau-217/Aβ42).

10. The composition of claim 1, wherein the magnesium bisglycinate chelate exhibits ≥90% solubility at physiological pH and provides elemental magnesium bioavailability ≥25% higher than magnesium oxide.

11. The composition of claim 1, further comprising a dosage stratification protocol wherein adults with baseline 25-hydroxyvitamin D levels <30 ng/ml receive the upper dosage ranges of components (1a)-(1 g).

12. A method for developing a dietary pattern directed nutritional supplement for cognitive health, comprising:
a) identifying neuroprotective dietary patterns by systematic literature analysis of human studies examining Mediterranean diet and MIND diet adherence in relation to cognitive outcomes, wherein dietary patterns are scored based on strength of neuroprotective evidence;
b) selecting target food groups abundant in the highest-scoring neuroprotective dietary patterns, said food groups including fish, leafy green vegetables, berries, nuts, tea, and olive oil;
c) generating a candidate nutrient list by comprehensive literature survey of nutrients abundant in the selected food groups and studied in context of cognition, neuroprotection, or neurodegeneration;
d) applying multi-criteria nutrient scoring to each candidate nutrient using five evaluation criteria consisting of observational/epidemiological evidence strength for cognitive benefit, clinical evidence quality and cognitive outcomes, putative biological mechanisms categorized by neuroprotective pathways, likelihood of intake inadequacy in target populations with suboptimal diets, and safety profile with established upper intake limits;
e) selecting final nutrients by ranking candidates by total scores across all criteria and choosing top-scoring nutrients that represent different food groups and diverse neuroprotective mechanisms; and
f) determining intake levels for selected nutrients based on diet-achievable concentrations below established upper limits that correspond to proportions found in high-adherence Mediterranean and MIND dietary patterns, wherein the method systematically develops nutritional supplements that mimic the complex nutrient profiles of evidence-based neuroprotective dietary patterns rather than targeting individual nutrient deficiencies or single botanical extracts, and wherein supplements developed by this method provide 12-25 distinct nutrients at nutritive intake levels equivalent to consuming at least 2-3 weekly servings of fish, daily leafy greens, daily nuts, and daily tea consumption.

13. The method of claim 12, further comprising administering herring roe oil comprising ≥70 wt % phospholipid-bound omega-3 fatty acids with a DHA:EPA weight ratio of 2.75:1 to 3.25:1, and wherein the phospholipid form provides a plasma omega-3 index increase or plasma omega-3 (DHA+EPA) increase of at least 1.25-fold compared to baseline.

14. The method of claim 12, further comprising administering RRR-alpha-tocopherol and gamma-tocopherol are co-formulated in a lipophilic softgel, providing a bioavailability enhancement of ≥40% compared to dry powder encapsulation as measured by plasma alpha-tocopherol/cholesterol ratio.

15. The method of claim 12, further comprising administering marigold flower extract standardized to provide lutein and zeaxanthin in their naturally occurring 5:1 weight ratio with ≥20% as meso-zeaxanthin.

16. The method of claim 12, further comprising administering polyphenolic components are processed via convection-drying, freeze-drying, or spray-drying at temperatures ≤40° C. to preserve bioactive aglycone forms, and wherein the catechins comprise ≥40 wt % epigallocatechin gallate (EGCG) with a polyphenol oxidase inhibition value ≤5%.

17. The method of claim 12, further comprising administering a multi-component delivery system comprising: (i) immediate-release or delayed-release capsules containing the B-vitamin and mineral components, (ii) delayed-release enteric capsules containing the polyphenolic components with gastric resistance for ≥2 hours at pH 1.2, and (iii) lipophilic softgels containing the omega-3, vitamin, and carotenoid components.

18. The method of claim 12, further comprising daily administration for 4-12 weeks to adults aged 50-90 years with MIND dietary screener scores ≤7.5 produces statistically significant (p<0.05) or near statistically significant (p≤0.10) improvements (by comparing measurements at baseline vs time points after daily administration is applied (or the reverse: comparing measurements while daily administration is applied vs the removal of daily administration) or by comparing measurements between daily administration and control groups) in at least three biomarkers (derived from blood or urine) selected from: omega-3 (docosahexaenoic acid (DHA), eicosapentaenoic acid (EPA), or DHA+EPA), omega-3 index, plasma omega-3 (DHA+EPA), total 25-hydroxyvitamin D (25 (OH) D), alpha-and gamma-tocopherol (vitamin E forms), lutein, zeaxanthin, nicotinamide (vitamin B3), cobalamin (vitamin B12 and other vitamin B forms), magnesium, catechins (including epigallocatechin gallate (EGCG), epigallocatechin (EGC), and epicatechin (EC)), quercetin, kaempferol, myricetin, anthocyanins (including parent anthocyanidins, which include cyanidin, delphinidin, malvidin, pelargonidin, peonidin, petunidin);

19. The method of claim 12, further comprising daily administration for 4-52 weeks to adults aged 50-90 years with MIND dietary screener scores ≤7.5 produces statistically significant (p≤0.05) or near statistically significant (p≤0.10) improvements (by comparing measurements at baseline vs time points after daily administration is applied (or the reverse: comparing measurements while daily administration is applied vs the removal of daily administration) or by comparing measurements between daily administration and control groups) in at least one biomarker (derived from blood or cerebrospinal fluid) selected from: ptau-217, ptau-181, total tau, amyloid beta 42 (Aβ42), amyloid beta 40 (Aβ40), glial fibrillary acidic protein (GFAP), neurofilament light chain (NfL), or pro-and anti-inflammatory cytokines implicated in brain aging and a number of neurodegenerative diseases (including (in their common scientific abbreviations) basic FGF, CTACK, eotaxin, G-CSF, GM-CSF, GRO-alpha, HGF, IFN-alpha2, IFN-gamma, IL-1alpha, IL-1beta, IL-1Ra, IL-2, IL-2Ralpha, IL-3, IL-4, IL-5, IL-6, IL-7, IL-8, IL-9, IL-10, IL-12 (p70), IL-12 (p40, IL-13, IL-15, IL-16, IL-17A, IL-18, IP-10, LIF, MCP-1 (MCAF), MCP-3, M-CSF, MIF, MIG, MIP-1alpha, MIP-1beta, beta-NGF, PDGF-BB, RANTES, SCF, SCGF-beta, SDF-1alpha, TNF-alpha, TNF-beta, TRAIL, and VEGF).

20. The method of claim 12, further comprising daily administration for 4-52 weeks to adults aged 50-90 years with MIND dietary screener scores ≤7.5 produces statistically significant (p≤0.05) or near statistically significant (p≤0.10) improvements (by comparing measurements ≤at baseline vs time points after daily administration is applied (or the reverse: comparing measurements while daily administration is applied vs the removal of daily administration) or by comparing measurements between daily administration and control groups) to the ratio of blood-derived biomarkers ptau-217 and Aβ42 (i.e. ratio of ptau-217/Aβ42).

21. The method of claim 12, further comprising administering magnesium bisglycinate chelate exhibiting ≥90% solubility at physiological pH and providing elemental magnesium bioavailability ≥25% higher than magnesium oxide.

22. The method of claim 12, further comprising the use of a dietary pattern directed nutritional supplementation regimen to induce changes or differences in the following measures in adults with suboptimal diet (as indicated by MeDi (or MEDAS) or MIND dietary screening scores), derived by comparing measurements at baseline vs time points after the method is applied (or the reverse: comparing measurements while method is applied vs the removal of method) or by comparing measurements between method and control groups:
a) the concentrations of nutrients and metabolites (in blood, urine, tissue, or otherwise derived from human samples) indicative of nutritional components abundant in neuroprotective diets, such as the MeDi or MIND diets, including: omega-3 (docosahexaenoic acid (DHA), eicosapentaenoic acid (EPA), DHA+EPA), total 25-hydroxyvitamin D (25 (OH) D), alpha-and gamma-tocopherol (vitamin E forms), lutein and zeaxanthin, nicotinamide (vitamin B3), cobalamin (vitamin B12 and other vitamin B forms), magnesium, catechins (including epigallocatechin gallate (EGCG), epigallocatechin (EGC), and epicatechin (EC)), quercetin, kaempferol, myricetin, anthocyanins (including parent anthocyanidins, which include cyanidin, delphinidin, malvidin, pelargonidin, peonidin, petunidin);
b) the concentrations of biomarkers of brain aging, Alzheimer's disease, or neurodegeneration, including ptau-217, ptau-181, total tau, amyloid beta 42 (Aβ42), amyloid beta 40 (Aβ40), glial fibrillary acidic protein (GFAP), neurofilament light chain (NfL), pro-and anti-inflammatory cytokines implicated in brain aging and a number of neurodegenerative diseases (including (in their common scientific abbreviations) basic FGF, CTACK, eotaxin, G-CSF, GM-CSF, GRO-alpha, HGF, IFN-alpha2, IFN-gamma, IL-1alpha, IL-1beta, IL-1Ra, IL-2, IL-2Ralpha, IL-3, IL-4, IL-5, IL-6, IL-7, IL-8, IL-9, IL-10, IL-12 (p70), IL-12 (p40, IL-13, IL-15, IL-16, IL-17A, IL-18, IP-10, LIF, MCP-1 (MCAF), MCP-3, M-CSF, MIF, MIG, MIP-1alpha, MIP-1beta, beta-NGF, PDGF-BB, RANTES, SCF, SCGF-beta, SDF-1alpha, TNF-alpha, TNF-beta, TRAIL, and VEGF), or ratios of such biomarkers (including ptau-217/Aβ42);
c) metabolomic (metabolic), lipidomic, proteomic, exposomic, or ionomic profiles in a manner that more closely resembles the respective profiles of high adherers of neuroprotective diets (e.g. MeDi or MIND), measured through either targeted or unbiased analyses, where metabolomic analyses may cover amino acid, cofactor/vitamin, nucleotide, carbohydrate, energy, lipid, and other metabolic classes;
d) clinical laboratory blood measurements, including LDL, HDL, triglycerides, total cholesterol, fasting glucose, HbA1C, and CRP;
e) scores of standardized cognitive tests, which may include rapid (3-15 min), self-administered digital/computerized cognitive screening tests, where cognitive domains tested may include memory, executive function, attention, visuospatial, and processing speed;
f) brain structure or imaging results, such as brain size, volume, specific area density, phosphorylated tau levels (for example, through positron-emission tomography (PET), brain amyloid plaque levels, brain volume or connectivity in total or in specific regions, gray matter, or white matter; and
g) wherein combinations of the above (22a)-(22f) indicating correlations between or among them and wherein changes or differences in items (22a)-(22f) could be derived by comparing measurements at baseline vs time points after the method is applied (or the reverse: comparing measurements while method is applied vs the removal of method) or by comparing measurements between method and control groups.

23. The method of claim 12, further comprising the use of a dietary pattern directed nutritional supplementation regimen to reduce risk of neurodegenerative disease (e.g. Alzheimer's disease, Parkinson's disease, Lewy body dementia) or delay progression of neurodegenerative disease processes or pathologies, as indicated by the method's induction of changes or differences in the following measures in adults, derived by comparing measurements at baseline vs time points after the method is applied, in a manner that reduces or maintains baseline levels of biomarkers of brain aging, Alzheimer's disease, or neurodegeneration, including ptau-217, ptau-181, total tau, amyloid beta 42 (Aβ42), amyloid beta 40 (Aβ40), glial fibrillary acidic protein (GFAP), neurofilament light chain (NfL), pro-and anti-inflammatory cytokines implicated in brain aging and a number of neurodegenerative diseases (including (in their common scientific abbreviations) basic FGF, CTACK, eotaxin, G-CSF, GM-CSF, GRO-alpha, HGF, IFN-alpha2, IFN-gamma, IL-1alpha, IL-1beta, IL-1Ra, IL-2, IL-2Ralpha, IL-3, IL-4, IL-5, IL-6, IL-7, IL-8, IL-9, IL-10, IL-12 (p70), IL-12 (p40, IL-13, IL-15, IL-16, IL-17A, IL-18, IP-10, LIF, MCP-1 (MCAF), MCP-3, M-CSF, MIF, MIG, MIP-1alpha, MIP-1beta, beta-NGF, PDGF-BB, RANTES, SCF, SCGF-beta, SDF-1alpha, TNF-alpha, TNF-beta, TRAIL, and VEGF), or ratios of such biomarkers (including ptau-217/Aβ42).

24. The method of claim 22, further comprising inducing its effects in adults ages 50-90 years of age, with suboptimal diet (as indicated by MeDi (or MEDAS) or MIND dietary screening scores).

25. The method of claim 23, further comprising inducing its effects in adults ages 50-90 years of age, with suboptimal diet (as indicated by MeDi (or MEDAS) or MIND dietary screening scores).

26. The method of claim 22, further comprising inducing its effects in adults with ptau-217 concentration or ptau-217/Aβ42 ratio elevated at baseline compared to mean levels of adults of similar age.

27. The method of claim 23, further comprising inducing its effects in adults with ptau-217 concentration or ptau-217/Aβ42 ratio elevated at baseline compared to mean levels of adults of similar age.
